(19) United States
(12) Patent Application Publication
(10) Pub. No.: US 2013/0138120 A1
(43) Pub. Date: May 30, 2013

(54) TISSUE ACQUISITION DEVICES AND METHODS

(71) Applicant: Barosense, Inc., Redwood City, CA (US)

(72) Inventors: Pablo Hambly, San Mateo, CA (US); Daniel Balbierz, Redwood city, CA (US); Samuel Crews, Woodside, CA (US); Bretton Swope, San Francisco, CA (US); David Cole, San Mateo, CA (US); Andrew Smith, San Francisco, CA (US)

(73) Assignee: Barosense, Inc., Redwood City, CA (US)

(21) Appl. No.: 13/747,450

(22) Filed: Jan. 22, 2013

Related U.S. Application Data
(63) Continuation of application No. 12/268,216, filed on Nov. 10, 2008, now abandoned.
Application No. 12/268,216 is a continuation-in-part of application No. 11/900,757, filed on Sep. 13, 2007, now abandoned.
(60) Provisional application No. 60/825,534, filed on Sep. 13, 2006.

(51) Int. Cl. A61B 17/10; A61B 17/068

(52) U.S. Cl. CPC A61B 17/10; A61B 17/068
USPC 606/139; 227/175.1

(57) ABSTRACT

A staple housing includes an array of staples each in a staple delivery position or “ready position” ready to be fired into target tissue. A staple driver is advanceable to drive the ready-position staples from the staple head into the tissue using staple pushers. During use, the staples in the ready positions are simultaneously fired into the target tissue using the staple pushers, forming an array of staples in the target tissue. After the array has been fired, one or more feed mechanisms within the staple housing advance a second group of staples from one or more staple storage locations into the ready positions in preparation for firing of the second group of staples.

### PRIORITY

This is a continuation of U.S. patent application Ser. No. 12/268,216, filed Nov. 10, 2008, which is a continuation-in-part of U.S. application Ser. No. 11/900,757, filed Sep. 13, 2007, which claims the benefit of U.S. Provisional Application No. 60/825,534, filed Sep. 13, 2006, all of which are incorporated by reference herein.

### BACKGROUND OF THE INVENTION

Surgical procedures used to modify the shape and/or size of a stomach are effective in reducing weight and resolving associated co morbidities. Unfortunately these surgical procedures are invasive and are associated with high levels of peri-operative and post operative complications.

Some procedures have been introduced which utilize natural body orifices for surgery to reduce the invasiveness of these procedures. Natural orifices include, but are not limited to the esophagus, anus and vagina. These procedures are less invasive by nature but have limitations as will be described below.

Natural orifice procedures have largely been directed at the gastrointestinal (GI) tract, but also include procedures which exit the GI tract, and perform surgeries normally done laparoscopically. Access to the peritoneal space for example can be accomplished by penetrating the stomach wall.

One primary means of stomach modification is by the use of surgical or laparoscopic staplers. These devices are able to surgically or laparoscopically appose multiple layers of tissue and connect them by use of multiple staple rows. Early procedures stapled across the outside of the stomach, which brought the mucosa of two sides of the stomach into apposition. There was, and is, a high rate of failure of these staple lines due to the nature of the GI tract. Staple line dehiscence was common and resulted in inadequate clinical results. The solution was to surgically staple the tissue and cut between the staple lines. This enabled edge to edge healing to occur, and provided for a robust tissue bridge. The separation/cutting of tissues is now common for surgical procedures such as Roux-En-Y Gastric Bypass, Sleeve Gastrectomy, and Vertical Banded Gastroplasty. However, less invasive procedures allowing stomach partitioning using natural orifice access are highly desirable.

Some existing procedures attempt to partition the stomach from the inside by connecting tissue within the stomach. To date these procedures have demonstrated a high failure rate. Improved devices and methods for creating robust stomach partitions using natural orifice access are disclosed in commonly owned U.S. application Ser. No. 11/900,757, filed Sep. 13, 2007, which was published as US 2008-0190989 and which is entitled ENDOSCOPIC PLICATION DEVICE AND METHOD.

As described in the '757 application, when an area of the stomach wall is drawn inwardly (bringing a two-layer “pinch” or fold of tissue toward the stomach interior), corresponding regions of serosal tissue on the exterior of the stomach are positioned facing one another. The applications discloses plication procedures in which two or more such areas or pinches of the stomach wall are engaged/grasped and drawn inwardly using instruments passed into the stomach via the mouth. The two or more pinches of tissue are held in complete or partial alignment with one another as staples or other fasteners are driven through the pinches, thus forming a four-layer tissue plication. Over time, adhesions formed between the opposed serosal layers create strong bonds that can facilitate retention of the plication over extended durations, despite the forces imparted on them by stomach movement.

One or more such plications may be formed for a variety of purposes. For example, plications may be used to induce weight loss by creating a barrier or narrowing within the stomach that will restrict the flow of food from the proximal stomach towards the distal stomach. For example, as discussed in the '757 application, a partition or barrier may be oriented to extend across the stomach, leaving only a narrow exit orifice through which food can flow from the proximal stomach to the distal stomach, or a similar antral barrier may be formed that will slow stomach emptying of stomach contents into the pylorus. In other cases, partitions or plications may be used to form a proximal pouch in the stomach or to reduce stomach volume to cause sensations of fullness after a patient eats relatively small quantities. Plications might also be used as a treatment for GERD to create a shield between the stomach and esophagus that will minimize reflux. Plications might also be used to close perforations in the stomach wall.

The present application describes an improved tissue acquisition instrument useful for engaging areas or pinches of tissue and supporting the engaged areas of tissue in complete or partial alignment as the areas are fastened to one another using fasteners, staples, sutures, etc.

### BRIEF DESCRIPTION OF THE DRAWINGS

FIG. 1 is a perspective view of an embodiment of a tissue acquisition device, showing the graspers in the retracted position;

FIG. 2A is a top perspective view of the acquisition head of the device of FIG. 1;

FIG. 2B is a perspective view of the acquisition head, taken in longitudinal cross-section, of the acquisition head of the device of FIG. 2A.

FIG. 3 is a perspective view of the acquisition head, showing one of the graspers in the deployed position;

FIG. 4 is a perspective view similar to FIG. 3, showing both graspers in the deployed position;

FIGS. 5A-5C are a series of perspective views of the distal portion of the acquisition head, showing both graspers in retracted, partially deployed, and fully deployed positions, respectively.

FIG. 6 is a perspective view of the proximal portion of the acquisition head, with the graspers in the retracted position.

FIG. 7 is a perspective view similar to FIG. 7 but with the housing eliminated to permit viewing of the graspers and associated features.

FIG. 8 is a perspective view of a distal portion of the acquisition head, with the graspers in the retracted position.

FIG. 9 is a perspective view of the proximal portion of the acquisition head, with the graspers in the deployed position.

FIG. 10 is a perspective view similar to FIG. 10 but with the housing eliminated to permit viewing of the graspers and associated features.

FIG. 11 is a perspective view of a distal portion of the acquisition head, with the graspers in the deployed position.

FIG. 12 is a top plan view of a proximal portion of the acquisition head, with the proximal portion of the housing shown in cross-section to permit viewing of the cable channels.

FIGS. 13A-13C are a sequence of transverse cross-section views of the acquisition head, schematically illustrating use of the device to acquire and grasp tissue.

FIG. 13D illustrates use of a tissue fastening device to secure the layers of tissue acquired and grasped in FIGS. 13A-13C.

### DETAILED DESCRIPTION

The present application describes a device and method for acquiring two or more areas or pinches of tissue and for supporting the acquired tissue until it has been fastened together using staples or other fasteners, or treated in some other way. Generally speaking, the disclosed device operates to acquire tissue using vacuum pressure, and to then hold or retain the acquired tissue in place using mechanical graspers. The device and method may be used in to procedure for joining tissue areas together to form tissue structures within, to remodel, or to partition a body cavity, hollow organ or tissue tract. The application will discuss the device and method in connection with use in the stomach for formation of plications such as for stomach partitioning or other purposes, although they may be used for applications other than stomach remodeling or partitioning.

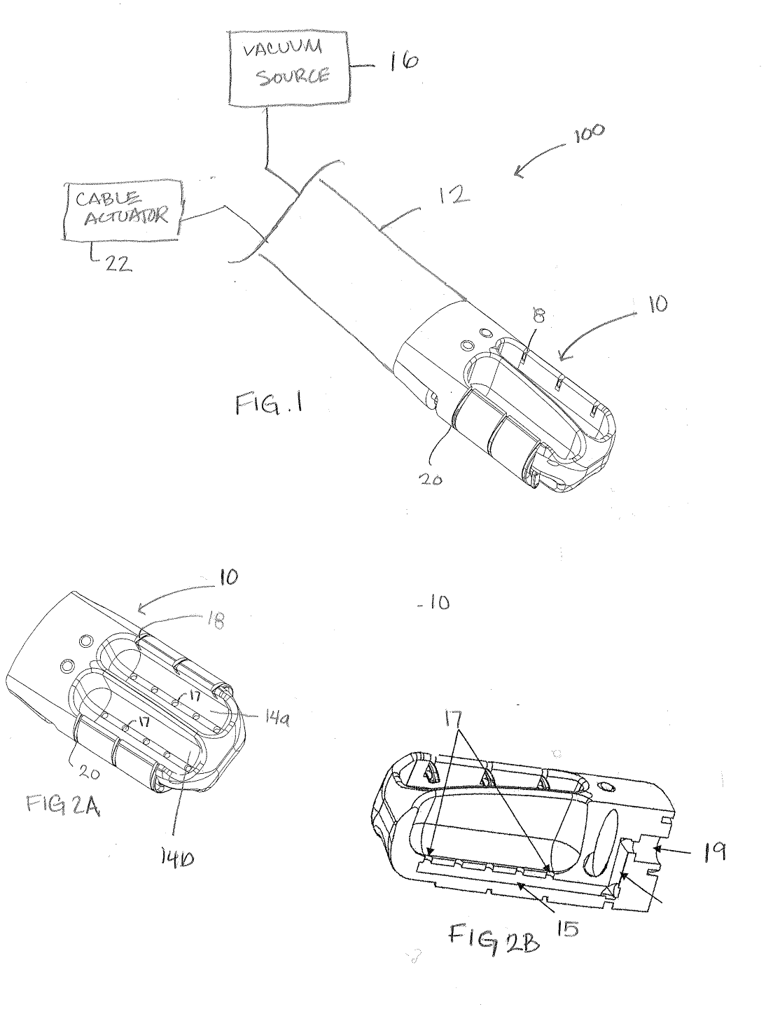

Referring to FIG. 1, an exemplary embodiment of a tissue acquisition device 100 includes a tissue acquisition head 10 positioned at the distal end of an elongate shaft 12. The shaft is of sufficient length to allow it to be advanced into the target body cavity (e.g. stomach) through a natural orifice (e.g. the mouth). While not shown in the drawing, the device 100 preferably includes articulation features allowing the head to be articulated to facilitate positioning of the head relative to target tissue.

Acquisition head 10 comprises a housing having a pair of vacuum chambers 14a, 14b. One or more vacuum sources 16 are fluidly coupled to the vacuum chambers 14a, 14b, preferably in a manner that allows a user to selectively apply vacuum pressure to the vacuum chambers 14a, 14b at different times. As shown in FIG. 2B, head 10 includes a pair of vacuum channels 15, each fluidly coupled with a plurality of holes 17 extending into an associated one of the vacuum chambers 14a, 14b. Vacuum pressure is applied to the vacuum channels 15 via openings 19 (see also FIG. 6) in the head 10. Each opening 19 is continuous with a lumen or channel through the shaft 12 to the vacuum source.

Controls on the vacuum source 16 or the shaft 12 allow a user to select which vacuum chamber 14a, 14b is to receive vacuum pressure at any given moment in the procedure. For example, each opening 19 in the handle may be continuous with a dedicated lumen in the shaft, where each lumen has a valve that may be opened to apply vacuum through that lumen to the associated one of vacuum chambers 20a, 20b. Alternatively, each opening 19 in the handle may be continuous with a dedicated lumen that is connected to its own source of vacuum pressure, so that vacuum pressure to a given vacuum chamber is initiated by activating the appropriate vacuum source.

The acquisition head further includes retention elements that function to mechanically engage a portion of the tissue that has been acquired by the vacuum chamber. Referring to FIGS. 3 and 4, channels 18 within the acquisition head 10 house advanceable and retractable graspers 20a, 20b that function as retention elements in the illustrated embodiment. Each grasper 20a, 20b shown includes a plurality of arcuate fingers 26 that extend into a corresponding one of the vacuum chambers 14a, 14b. In alternate embodiments, the graspers can have a variety of other configurations, including those that do not extend into the vacuum chamber but that instead clamp an outer section of the tissue drawn into the chamber against the exterior surface of the acquisition head.

A least one actuator 22 is positioned on the handle 12 (FIG. 1) allowing for independent control of each of the graspers 20a, 20b. During the course of a procedure, a user may use the actuator(s) 22 to first deploy the grasper 20b as in FIG. 3 to engage tissue drawn into vacuum chamber 14b, and to later advance the graspers 20a (FIG. 4) to engage a second pinch of tissue subsequently drawn into the vacuum chamber 14a.

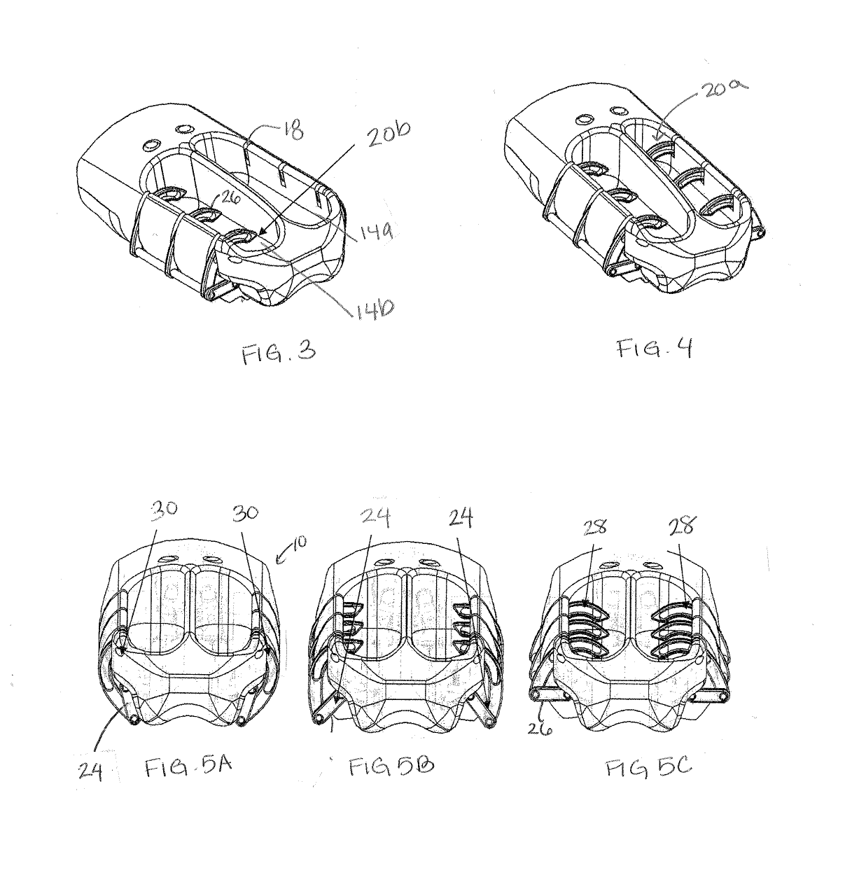

FIGS. 5A-5C illustrate advancement of the graspers 20a, 20b from the retracted position (FIG. 5A), to a semi-deployed position (FIG. 5B), to a fully deployed position (FIG. 5C). Each of the fingers 26 includes a curved guide channel 28 (most visible in FIGS. 5B and 5C). A pair of guide pins 30 (only the distal ends of which are visible in FIGS. 5A-5C) extend longitudinally through each side of the head 10. Each of the guide pins 30 extends through the guide channels on that side of the head 10. As the fingers 26 move to the deployed position, their guide channels slide over the guide pins 30, ensuring that the free ends of the fingers track downwardly into the corresponding vacuum chamber.

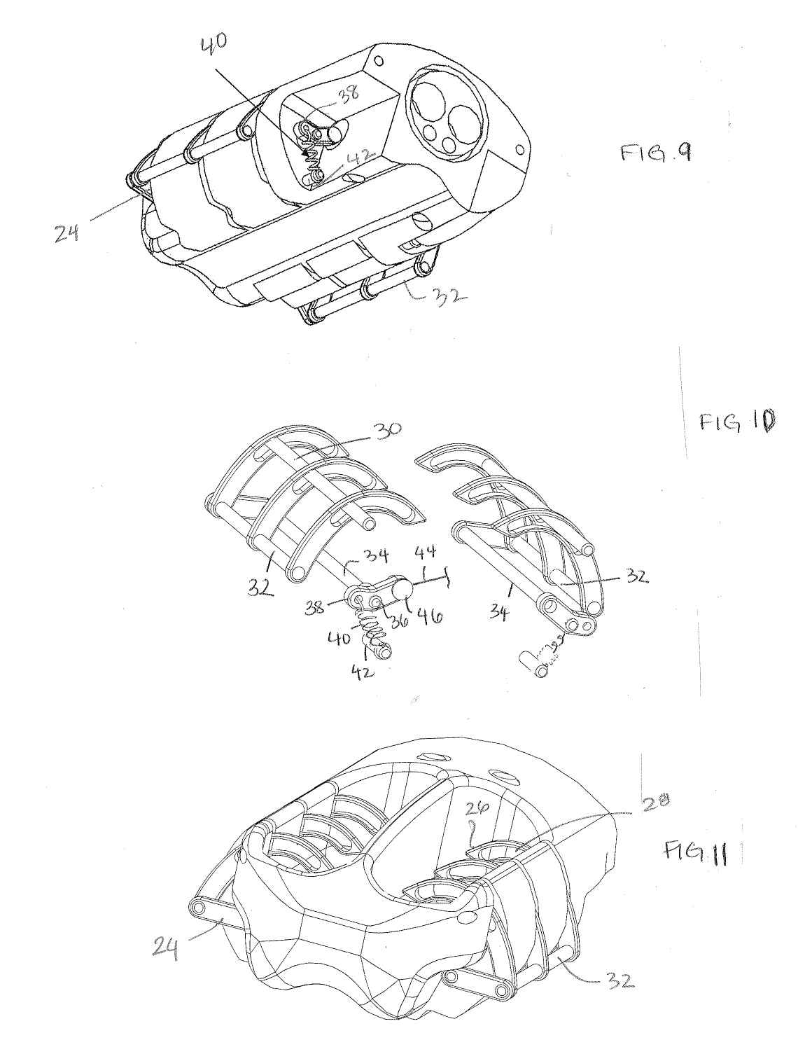

FIGS. 5A-5C further illustrate movement of drive links 24, which pivot laterally outwardly to drive the graspers 20a, 20b to the deployed position. The mechanism for driving the links 24 for advancing and retracting the graspers 20a, 20b will next be described with respect to FIGS. 6-11. Attention is first directed to FIGS. 6 and 10, in which the housing of the head 10 has been omitted to permit clear viewing of the graspers and associated features. A longitudinally extending pin 32 connects the fingers 26 of each grasper 20a, 20b. Each of the drive links 24 has a first end coupled to the distal end of one of the pins 32, and a second end coupled to an arm rotator pin 34 which is oriented longitudinally relative to the housing.

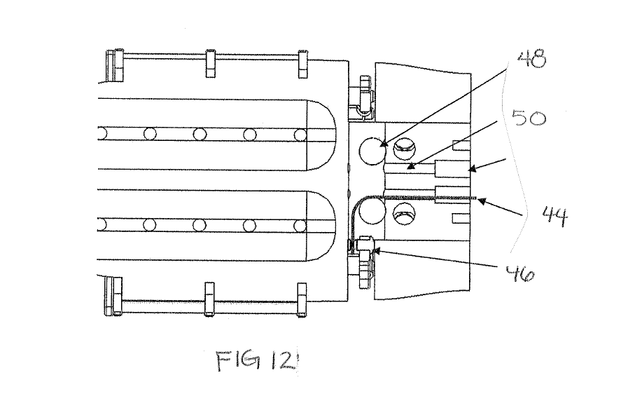

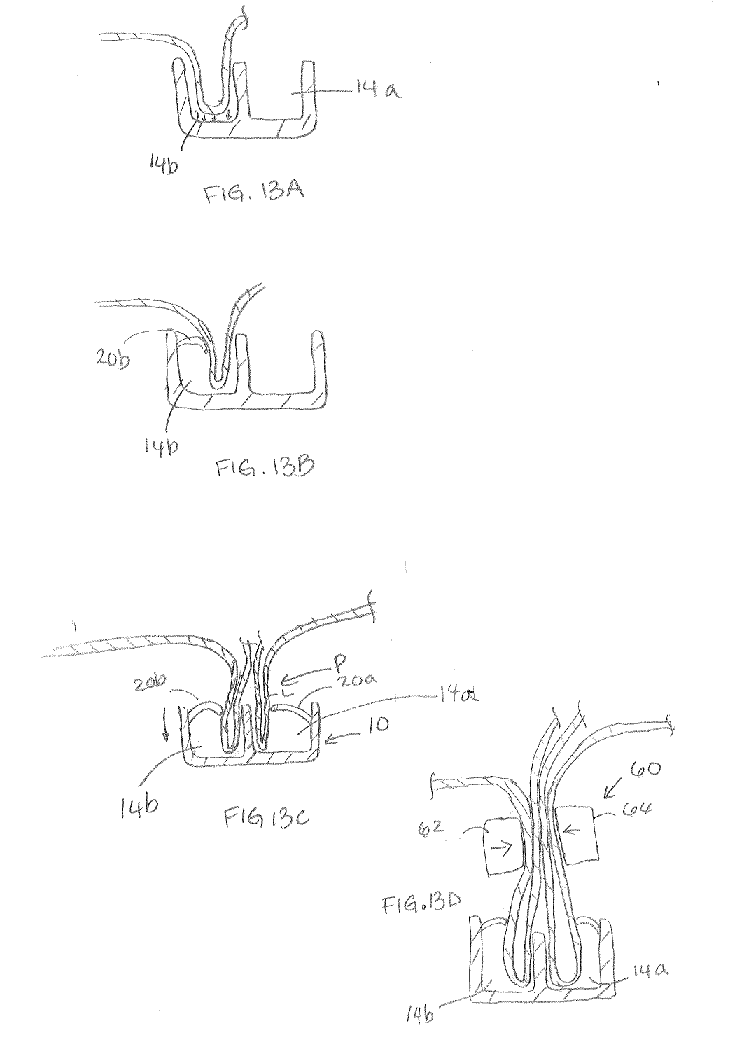

A cable link 38 is attached to the proximal end of the arm rotator pin 34, at pivot location 36. Cable link has ends pivotable about the pivot location 36. A spring 40 extends between one end of the cable link 38 and a pin 42 mounted to the housing of the head 10. The other end of the cable link 38 includes an end pin 46 to which a pull cable 44 is secured. Referring to FIG. 12, a pull cable 44 extends from end pin 46, around a cylindrical cable guide 48, into a cable channel 50 (also see FIG. 6) and through the shaft 12 (FIG. 1) where it is coupled to actuator 22.

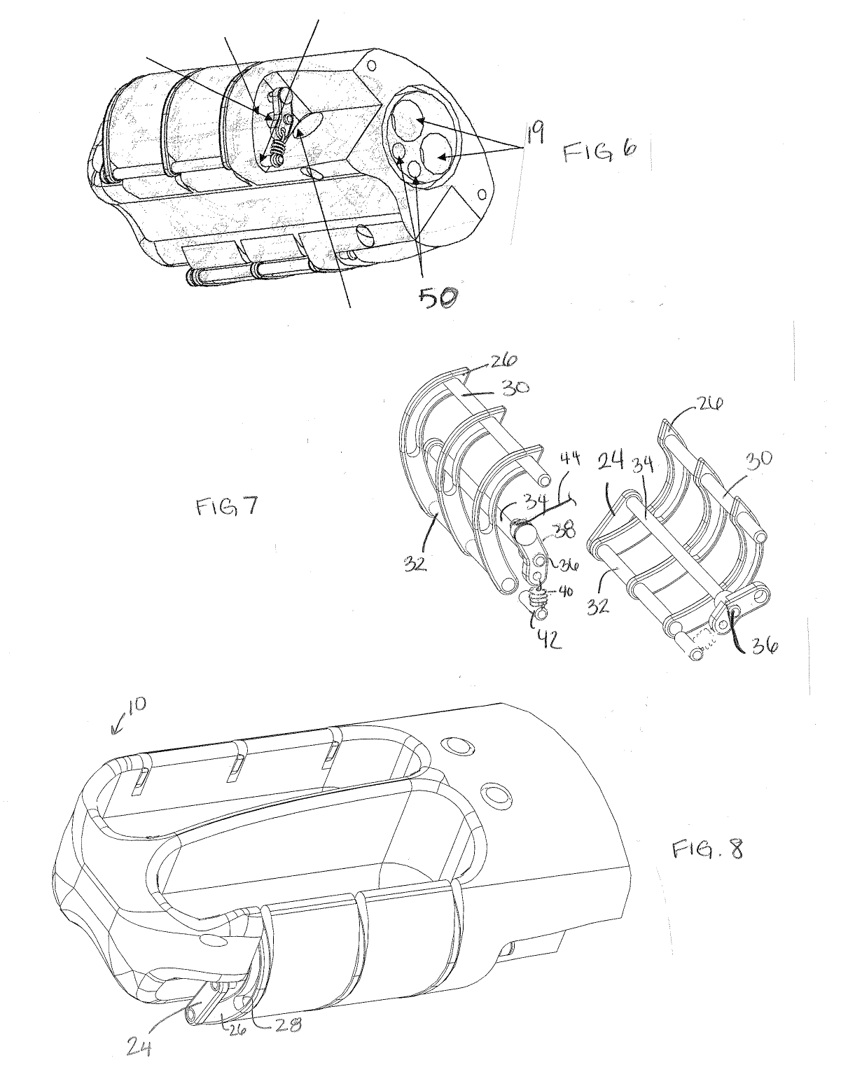

To deploy one of the graspers 20a, 20b, actuator 22 is manipulated to pull the cable 44 associated with the grasper to be deployed. Tension on the cable 44 rotates the cable link 38 about pivot 36 from the position shown in FIG. 7 to the position shown in FIG. 10. Rotation of the cable link 38 rotates the arm rotator pin 34, thus causing link 24 to pivot laterally outwardly from the position shown in FIG. 8 to the position shown in FIG. 11. This movement of the link 24 pivots the pin 32 laterally outwardly, and thereby advances the attached fingers 26 to the deployed position.

The spring 40 serves to bias the cable link 38 in the position shown in FIG. 7, thus keeping the graspers 20a, 20b biased in the retracted position. It can be seen by comparing FIGS. 7 and 10 that when the cable link 38 is rotated by the cable 44, the spring expands from its resting position to a position in tension. When the actuator 22 is caused to release the tension on the cable 44, the spring returns to its resting state, thereby returning the graspers to the retracted position.

FIGS. 13A-13D schematically illustrate use of the acquisition device to place two two-layer folds of tissue in apposition for fastening together using a tissue fastener such as a stapler, clip applier, suture device etc, although the acquisition device may be used for procedures as well.

During use of the acquisition device, the head 10 of the device 100 is introduced into a patient (e.g. into the stomach through an endogastric overtube) and advanced towards tissue to be acquired. A first one of the vacuum chambers 14b is positioned adjacent to the target tissue, and the vacuum source is activated relative to that chamber, thus drawing the target tissue into the chamber as shown in FIG. 13A. Once a sufficient volume of tissue has been drawn into the chamber, the actuator is used to drive the grasper 20b into the chamber 14b as shown in FIG. 13B. When the tissue has been securely acquired by the first grasper, vacuum pressure in chamber 14b is released and the vacuum head (with the first tissue pinch securely engaged in chamber 14b) is repositioned to position the vacuum chambers 14a at a second area of target tissue. Vacuum is again initiated to draw tissue into the second chamber 14a. After the desired volume of tissue has been drawn into the chamber 14a, grasper 20a is advanced to retain the tissue within chamber 14a. At this time the vacuum may again be released.

Once tissue has been secured in each chamber as shown in FIG. 13C, the acquisition head 10, with the two tissue pinches retained by its graspers, is manipulated to place the layers L of the tissue pinches or folds P in tension. A fastening instrument 60 is positioned as shown in FIG. 13D to drive fasteners transversely through the four layers of tissue comprising the acquired tissue pinches. Fastening instrument 60 may be a stapler having a cartridge 62 and anvil 64. Commonly owned U.S. Pat. No. 7,934,631, entitled MULTI-FIRE STAPLING SYSTEMS AND METHODS FOR DELIVERING ARRAYS OF STAPLES, and filed on the same date as the present application, describes a stapler that may be used for this purpose. The tissue acquisition device may be part of a tissue reconfiguration or partitioning system that also includes the disclosed stapler, or any of a variety of mechanisms for applying a fastening element (e.g. clips, sutures, staplers, two-part fasteners etc.) to the acquired tissue.

It should be recognized that a number of variations of the above-identified embodiments will be obvious to one of ordinary skill in the art in view of the foregoing description. Moreover, features of the disclosed embodiments may be combined with one another and with other features (including those taught in the prior applications referenced herein) in varying ways to produce additional embodiments. Accordingly, the invention is not to be limited by those specific embodiments and methods of the present invention shown and described herein. The applications and methods listed are not limited to the treatment of diseases or procedures listed. Modifications of the above described methods and tools and variations of this invention that are obvious to those of skill in the art are intended to be within the scope of this disclosure.

Any and all patents, patent applications and printed publications referred to above, including those relied upon for purposes of priority, are incorporated herein by reference.

It is claimed:

1. A tissue acquisition device for intraorally fastening a fastened tissue fold having four or more tissue layers, comprising:
an acquisition head including at least a first and a second vacuum chamber, each of the first and second vacuum chambers capable of being coupled to at least one vacuum source, the head being positionable adjacent target tissue to draw at least a portion of the target tissue into the vacuum chambers;
at least a first and a second retention element coupled to the acquisition head, the retention elements being moveable from a first position to a second position, the first retention element in the second position is positioned to retain a first pinch of tissue in contact with the first vacuum chamber without piercing the tissue and the second retention element in the second position is positioned to retain a second pinch of tissue in the second vacuum chamber without piercing the tissue; and
a fastening instrument for stapling two or more pinches of tissue to form the fastened tissue fold having four or more tissue layers.

2. The acquisition device of claim 1, wherein the first retention element in the second position extends at least partially into the first vacuum chamber, and the second retention element in the second position extends at least partially into the second vacuum chamber, to retain tissue drawn into the first and second vacuum chambers, respectively.

3. The acquisition device of claim 1, wherein the first and second retention elements includes a plurality of fingers, the fingers extending at least partially into the first and second vacuum chambers when the retention elements are in the second position.

4. The acquisition device of claim 1, wherein the first and second vacuum chambers are fluidly independent of one another.

5. The acquisition device of claim 1, wherein the first and second retention elements are independently moveable between the first and second positions.

6. The acquisition device of claim 1, wherein the first and second retention elements are biased in the first position, and moveable against the bias to the second position.

7. The acquisition device of claim 1, further including an elongate shaft dimensioned to access a patient's stomach, the shaft having a proximal end and a distal end wherein the acquisition head is positioned on the distal end of the elongate shaft.

8. The device of claim 1, wherein the first retention element includes a plurality of arcuate fingers that extend into the first vacuum chamber to retain the first pinch of tissue in the first vacuum chamber and the second retention element includes a plurality of arcuate fingers that extend into the second vacuum chamber to retain the second pinch of tissue in the second vacuum chamber.

9. The device of claim 1, further comprising:
a first drive link operatively connected to the first retention element; and
a second drive link operatively connected to the second retention element;
the first and second drive links being operable to pivot laterally outwardly to drive the first and second retention elements, respectively, into the second position.

10. The device of claim 1, wherein the first vacuum chamber is capable of being coupled to a first vacuum source and the second vacuum chamber is capable of being coupled to a second vacuum source.

11. The device of claim 1, wherein the first vacuum chamber is capable of being coupled to a first vacuum source and the second vacuum chamber is capable of being coupled to a second vacuum source.

12. A tissue attachment system for forming a fastened tissue fold having four or more tissue layers, comprising:
a tissue acquisition device comprising
an acquisition head having at least a first and a second vacuum chamber, the first and second vacuum chambers each capable of being attachable to at least one vacuum source,
a first retention element moveable between a first position and a second position, the first retention element in the second position is positioned to retain a first pinch of tissue drawn at least partially into the first vacuum chamber without piercing the first pinch of tissue;
a second retention element moveable between a first position and a second position, the second retention element in the second position is positioned to retain a second pinch of tissue drawn at least partially into the second vacuum chamber without piercing the second pinch of tissue; and
a tissue fastening device positionable to advance at least one tissue fastener through the first and second pinch of tissue retained by the first and second retention elements thereby to form a tissue plication having four layers of tissue at the fastener.

13. The tissue attachment system of claim 12, wherein the first and second retention elements are independently moveable between the first and second positions.

14. The tissue attachment system of claim 12, wherein the fastener includes at least one staple.

15. The tissue attachment system of claim 12, wherein the fastener includes at least one suture.

16. The tissue attachment system of claim 12, wherein the fastener includes at least one clip.

17. The tissue attachment system of claim 12, wherein the fastener includes at least one two-part fastener.

18. The tissue attachment system of claim 12, wherein the first retention element includes a plurality of arcuate fingers that extend into the first vacuum chamber to retain the first pinch of tissue in the first vacuum chamber and the second retention element includes a plurality of arcuate fingers that extend into the second vacuum chamber to retain the second pinch of tissue in the second vacuum chamber.

19. The tissue attachment system of claim 12, further comprising:
a first drive link operatively connected to the first retention element; and
a second drive link operatively connected to the second retention element;
the first and second drive links being operable to pivot laterally outwardly to drive the first and second retention elements, respectively, into the second position.

20. A method of acquiring target tissue, comprising:
intraorally positioning an acquisition device at a first portion within a patient's stomach, the device having at least a first and a second vacuum chamber, the first and second vacuum chambers each capable of being coupled to at least one vacuum source and a first and second retention element moveable between a first and a second position;
drawing a first tissue fold into the first vacuum chamber by applying suction to the first chamber from the at least one vacuum source;
moving the first retention element from the first position to the second position where at least a portion of the first retention element is within the first vacuum chamber, whereby in the second position, the first retention element is in contact with the tissue fold drawn into the first vacuum chamber to retain the tissue fold in the first vacuum chamber without piercing the tissue;
positioning the acquisition device at a second portion of the stomach while retaining the first pinch in the first vacuum chamber;
drawing a second tissue fold into the second vacuum chamber by applying suction to the second chamber from the at least one vacuum source;
moving the second retention element from the first position to the second position where at least a portion of the second retention element is within the second vacuum chamber, whereby in the second position, the second retention element is in contact with the tissue fold drawn into the second vacuum chamber to retain the tissue fold in the second vacuum chamber without piercing the tissue; and
fastening the first and second tissue folds to form a tissue plication having four layers of tissue at a fastener point.

21. The method of claim 20, wherein the method further includes, prior to fastening the first and second tissue folds, manipulating the acquisition device to place tissue of the tissue folds under tension.
